(19) United States
(12) Patent Application Publication
(10) Pub. No.: US 2023/0348486 A1
(43) Pub. Date: Nov. 2, 2023

(54) NEAR-INFRARED EMITTING PORPHYRIN COMPOUND AND PREPARATION METHOD AND USE THEREOF

(71) Applicant: PEKING UNIVERSITY, Beijing (CN)

(72) Inventors: JUNLONG ZHANG, Beijing (CN); YINGYING NING, Beijing (CN); ZISHU YANG, Beijing (CN); BINGWU WANG, Beijing (CN)

(21) Appl. No.: 17/758,973

(22) PCT Filed: Dec. 23, 2020
(86) PCT No.: PCT/CN2020/138669

§ 371 Date: Jul. 17, 2022

(51) Int. Cl. C07D 491/22; A61P 35/00; C07D 498/22

(52) U.S. Cl. CPC C07D 491/22; A61P 35/00; C07D 498/22

(57) ABSTRACT

The present invention provides a porphyrin compound and its preparation method and uses, and also provides a pharmaceutical composition comprising the porphyrin compound as the active ingredient. The porphyrin compound according to the present invention has a novel modified structure, and can be derived and modified at multiple sites to achieve the biocompatibility modification and functional changes. The porphyrin compound has the absorption wavelength located in the near infrared region, which is contributed to realize the deeper tissue penetration and excellent photodynamic therapy activity.

### TECHNICAL FIELD

The present invention relates to the field of photodynamic therapy and bioimaging, in particular to a porphyrin-based macrocycle organic compound for photodynamic therapy and deep red to near infrared imaging, and to its preparation method and biological applications as well.

### BACKGROUND ART

Photodynamic therapy is a non-invasive therapeutic modality that has been used for the treatment of cancer, eye diseases and skin diseases. To achieve the purpose of treatment, photodynamic therapy involves the administration of a non-toxic photosensitizer into the body followed by irradiation of selected light with a wavelength suitable for exciting the photosensitizer on the location of lesion where the photosensitizer has arrived through the blood circulation and selectively aggregated, thereby inducing the excited photosensitizer to release reactive oxygen species which can kill diseased cells via directly killing, destroying blood vessels in diseased tissues and triggring immune stress, or the like.

For photodynamic therapy, the infrared wavelength of the therapeutic drug(i.e. photosensitizer) is a critical factor that will significantly affect its phototoxicity and tissue penetration depth and subsequently affect the clinical effect of photodynamic therapy. The therapeutic drug with longer infrared wavelength can achieve much deeper tissue penetration, which is useful to the preformance of the drug’s phototoxicity.

Currently, most of the photodynamic drugs in clinical applications are administered by injection, and the drugs will targeted aggregate on the location of lesion. During the aggregation of the drugs, problems such as drug metabolism failure, low concentration on the location of lesion, and increased skin photosensitivity of the patient’s whole body may occur with the systemic circulation.

Therefore, for photodynamic therapy, there is still a need for photosensitizer compounds with stronger phototoxicity and longer infrared wavelength, especially those which can be administered in vitro so as to reduce the current clinical side effects of photodynamic therapy.

In views of the above-mentioned problems, the present inventors have made a great effort and provided a phototoxic deep red to near-infrared emitting porphyrin compound and its preparation method and use.

### SUMMARY

In order to solve the problems as described above, the present invention provides a porphyrin compound with novel modified structure. The porphyrin compound of the present invention can be derived and modified at multiple sites to achieve biocompatibility modification and functional changes, and it has longer infrared wavelength, stronger phototoxicity, deeper tissue penetration depth, excellent photodynamic therapy activity, and futher has fluorescent labeling and deep red-near infrared imaging functions.

One object of the present invention is to provide a porphyrin compound or its pharmaceutically acceptable salt, solvate, non-covalent complex or prodrug, comprising the following structure:

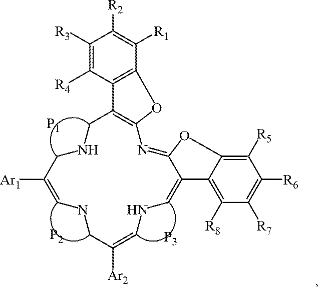

wherein, P₁, P₂ and P₃ are each independently 5-membered ring residues, the terminal carbon atoms of which are connected to N atoms on the 16-membered ring to form a ring; Ar₁ and Ar₂ are substituted or unsubstituted phenyl, aryl or heterocyclic aryl groups; R₁, R₂, R₃, R₄, R₅, R₆, R₇ and R₈ are substituents on benzene rings of the porphyrin compound, each independently selected from any one of hydrogen, halogen, nitro, hydroxyl, amino, mercapto, carboxyl, sulfonate group, phosphate group, cyano group, amide group, C₁₋₈ alkyl substituted amino group, substituted or unsubstituted C₁₋₁₂ alkyl group, substituted or unsubstituted C₁₋₈ alkoxy group, substituted thiol group, C₁₋₈ alkyl phosphate group, C₁₋₈ alkyl carboxyl group, C₁₋₈ alkyl sulfonate group, C₃₋₆ alkenyl alkyl group, C₂₋₆ alkenyl group, C₂₋₆ alkynyl alkyl group, C₂₋₆ alkynyl group, C₂₋₆ alkenyloxy group, C₂₋₆ alkynyloxy group, and C₁₋₅ alkanoyl.

Another object of present invention is to provide a method for preparing the porphyrin compound, comprising that:

the porphine lactone is reacted with an inorganic base or an organic base in a first organic solvent at 80-200° C. under inert ambient, to produce a first product, which is a porphyrin compound; and

optionally, the first product is subjected to oxidation, reduction or water-soluble modification reaction in a second organic solvent, to produce a second product, which is also a porphyrin compound.

Still another object of the present invention is to provide a pharmaceutical composition comprising the porphyrin compound as an active ingredient, wherein the composition further comprises pharmaceutically acceptable excipients; the composition is administered by injection or external use; and the amount of the active ingredient is 0.01 mg-20 g per unit dosage of the composition.

Yet still another object of the present invention is to provide a use of the porphyrin compound or its pharmaceutically acceptable salt, solvate, non-covalent bond complex or prodrug, and the pharmaceutical composition containing the porphyrin compound as the active ingredient for photodynamic therapy, preferably, a use in preparation of drugs for treating subcutaneous tumors including melanoma, sarcoma, fibroma, neurofibroma, lipoma, acne, schwannoma, hemangioma, leiomyoma and lymphangioma.

Further still another object of the present invention is to provide a use of the porphyrin compound or its pharmaceutically acceptable salt, solvate, non-covalent bond complex or prodrug, and the pharmaceutical composition containing the porphyrin compound as the active ingredient for fluorescence-labeling and infrared or fluorescence imaging in deep red to near infrared region.

The porphyrin compound and its preparation method and use accordin to the present invention have the following beneficial effects:
(1) The absorption wavelength of the porphyrin compound is located in the near-infrared region, which is contributed to achieve deeper tissue penetration depth and good photodynamic therapy activity, so that, through multi-cell line and in vivo photodynamic therapy experiments, it has been demonstrated that, the porphyrin compound according to the present invention has a better photodynamic therapy effect compared to the conventional photodynamic therapy drugs.
(2) The porphyrin compound has a novel modified structure, and can be derived and modified at multiple sites to achieve biocompatibility modification and functional changes due to different application requirements.
(3) The absorption and emission wavelength of the porphyrin compound fall within the visible to near-infrared region, which allows it to be excited by near-infrared light, so that it can be used in fluorescent labeling, especially in infrared or fluorescent imaging.
(4) The porphyrin compound has high phototoxicity, good biocompatibility, and high safety, and can be injected or externally administered via skin to treat subcutaneous tumors.

### DESCRIPTION OF FIGURES

FIG. 1 shows the absorption and emission spectra of the compounds obtained in Example 1-4 of the present invention;

FIG. 2 shows the results of the animal experiment in Experimental Example 2 of the present invention, wherein the left graph shows the change of tumor size, and the right graph shows the change of weight;

FIG. 3 shows the result of the in vivo fluorescence imaging experiment in Experimental Example 4 of the present invention.

### EMBODIMENTS

The present invention will be further described in detail below through the drawings and embodiments. Through these descriptions, the characteristics and advantages of the present invention will become clearer.

The term “exemplary” herein means “serving as an example, embodiment, or illustration.” Any embodiment described herein as “exemplary” need not be construed as being superior to or better than other embodiments. Although various aspects of the embodiments are shown in the drawings, unless otherwise noted, the drawings are not necessarily drawn to scale.

Hereinafter, the present invention will be described in details.

The present invention provides a porphyrin compound or its pharmaceutically acceptable salt, solvate, non-covalent complex or prodrug, comprising the following structure:

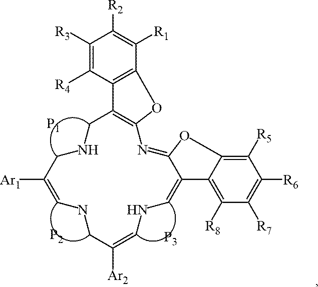

wherein, P₁, P₂ and P₃ are each independently 5-membered ring residues, the terminal carbon atoms of which are connected to N atoms on the 16-membered ring to form a ring; Ar₁ and Ar₂ are substituted or unsubstituted phenyl, aryl or heterocyclic aryl groups; R₁, R₂, R₃, R₄, R₅, R₆, R₇ and R₈ are substituents on benzene rings of the porphyrin compound, each independently selected from any one of the group consisting of hydrogen, halogen, nitro, hydroxyl, amino, mercapto, carboxyl, sulfonate group, phosphate group, cyano group, amide group, C₁₋₈ alkyl substituted amino group, substituted or unsubstituted C₁₋₁₂ alkyl group, substituted or unsubstituted C₁₋₈ alkoxy group, substituted thiol group, C₁₋₈ alkyl phosphate group, C₁₋₈ alkyl carboxyl group, C₁₋₈ alkyl sulfonate group, C₃₋₆ alkenyl alkyl group, C₂₋₆ alkenyl group, C₂₋₆ alkynyl alkyl group, C₂₋₆ alkynyl group, C₂₋₆ alkenyloxy group, C₂₋₆ alkynyloxy group, and C₁₋₅ alkanoyl.

In the context, unless otherwise stated, the halogen is selected from one or more of F, Cl, Br and I.

The alkyl group includes a linear, branched or cyclic saturated hydrocarbon group, preferably, is a C₁₋₆ alkyl, such as methyl, ethyl, propyl, isopropyl, butyl.

C₁₋₈ is a hydrocarbon chain with 1-8 carbon atoms, and similarly, C₃₋₆ is a hydrocarbon chain with 3-6 carbon atoms.

The alkyl-substituted amino group is an amino group substituted by the aforementioned alkyl group, preferably a C1-C6 alkyl-substituted amino group, such as methylamino, ethylamino, dimethylamino, and diethylamino.

The alkoxy group is an alkyloxy ether group, preferably a C1-C4 alkoxy group, such as methoxy group and propoxy group.

The substituted thiol group is a thiol group substituted by one of C₁₋₈ alkyl, glucosyl, mannose, fructose, galactose, ribose, and xylose, more preferably by C1-C4 alkyl, glucosyl, fructose, galactose, ribose, such as methylthio, ethylthio, and propylthio.

The alkyl phosphate group is an alkyl substituted phosphate group, preferably a C1-C4 alkyl substituted phosphate group, such as methyl phosphate group, ethyl phosphate group, and propyl phosphate group.

The alkyl sulfonate group is an alkyl-substituted sulfonate group, preferably a C1-C4 alkyl-substituted sulfonate group, such as methanesulfonate group, ethanesulfonate group, and propanesulfonate group.

The alkyl carboxyl group is a alkyl-substituted carboxyl group, such as acetoxyl group.

The alkenyl is a linear, branched or cyclic alkenyl group. The alkenyl alkyl group is an alkyl group containing the aforementioned alkenyl group. The alkenyloxy group is an oxyether group containing the aforementioned alkenyl group.

The alkynyl is a linear, branched or cyclic alkynyl group. The alkynyl alkyl group is an alkyl group containing the aforementioned alkynyl group. The alkynyloxy group is an oxyether group containing the aforementioned alkynyl group.

The alkanoyl is an acyl group containing the aforementioned alkyl group.

The aryl group is an aromatic ring containing a phenyl group, typically, is benzene, naphthalene, anthracene or phenanthrene, preferably benzene, naphthalene.

The heteroaryl group is a monocyclic or polycyclic aromatic group containing heteroatom(s), preferably 5-10 membered ring. The polycyclic aromatic group may be a double monoaromatic ring, a benzo monoaromatic ring or a condensed aromatic ring group. For example, the aryl group may be furan, pyridine, thiophene, imidazole, pyrrole, pyridazine, pyrazine, benzopyrrole, benzofuran, benzisoquinoline, pyrazinopyridazine, or the like.

Preferably,

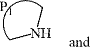

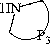

formed by connecting P₁ and P₃ with N atoms on 16-membered ring of the porphyrin compound are substituted or unsubstituted pyrrole rings. The substituents on the pyrrole rings may be one or more selected from the group consisting of halogen, hydroxyl, mercapto, amino, carboxyl, nitro or their combination.

Further,

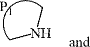

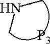

may be each independently selected from one of the group consisting of

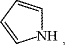

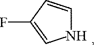

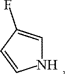

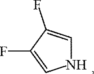

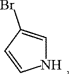

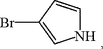

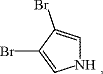

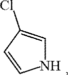

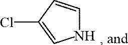

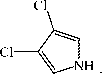

In some preferred embodiments, those formed by connecting P₁ and P₃ with N atoms on 16-membered ring of the porphyrin compound are unsubstituted pyrrole rings.

Preferably,

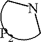

formed by connecting P₂ with N atom on 16-membered ring of the porphyrin compound is selected from one of the group consisting of

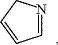

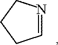

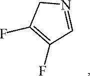

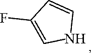

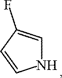

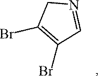

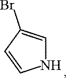

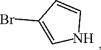

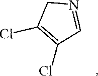

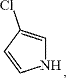

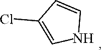

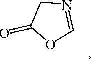

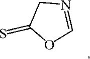

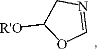

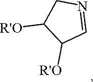

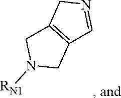

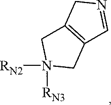

wherein, R′ is selected from the group consisting of hydrogen, trimethylaminoethyl; R_{N1}, R_{N2}, and R_{N3} are each independently selected from the group consisting of hydrogen, C₁₋₄ alkyl, C₁₋₄ alkoxy, halogen-substituted C₁₋₄ alkyl, C₁₋₄ alkyl-substituted amino, or C₁₋4 alkylthiol.

Further,

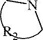

is selected from one of the group consisting of

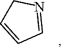

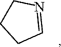

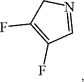

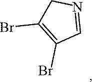

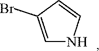

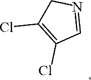

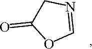

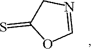

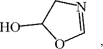

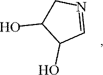

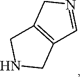

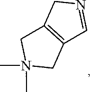

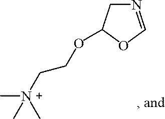

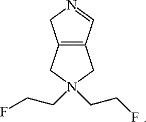

Preferably,

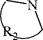

is selected from one of the group consisting of

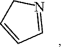

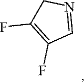

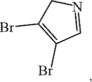

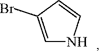

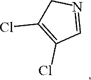

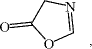

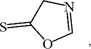

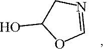

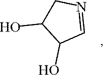

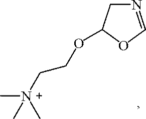

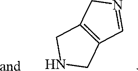

In some preferred embodiments,

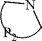

is

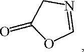

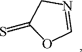

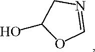

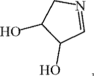

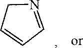

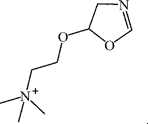

Preferably, Ar₁ and Ar₂ are substituted phenyl groups containing one or more substituents at sites selected from any one of the group consisting of the following:

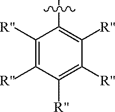

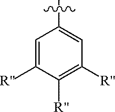

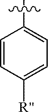

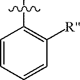

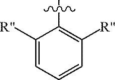

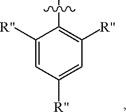

wherein, the substituent groups R″ of Ar₁ and Ar₂ are each independently any one or more selected from the group consisting of hydrogen, halogen, nitro, hydroxyl, mercapto, C₁₋₆ alkyl, C₁₋₆ alkoxy, halogen substituted C₁₋₆ alkyl, C₁₋₆ alkyl substituted amino group, substituted thiol group, phosphate group, C₁₋₆ alkyl phosphate group, carboxyl, C₁₋₆ alkyl carboxyl, sulfonate group, and C₁₋₆ alkyl sulfonate group.

Further, the substituent groups R″ of Ar₁ and Ar₂ are one or more selected from the group consisting of hydrogen, F, Cl, Br, nitro, hydroxyl, thiol, methyl, glucosyl substituted thiol, fructosyl substituted thiol, galactosyl substituted thiol, ribosyl substituted thiol, amino, trimethylamino, triethylamino, carboxyl, and sulfonate group.

In some preferred embodiments, the substituent groups R″ of Ar₁ and Ar₂ are one or more selected from the group consisting of F, Cl, Br, hydrogen, glucosyl substituted thiol, trimethylamine, and sulfonate group.

In some preferred embodiments, Ar₁ and/or Ar₂ are

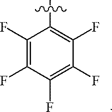

In some preferred embodiments, Ar₁ and/or Ar₂ are

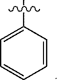

In some preferred embodiments, Ar₁ and/or Ar₂ are

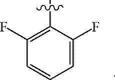

In some preferred embodiments, Ar₁ and/or Ar₂ are

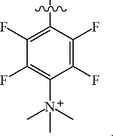

In some preferred embodiments, Ar₁ and/or Ar₂ are

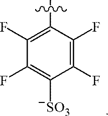

In some embodiments, Ar₁ and/or Ar₂ are

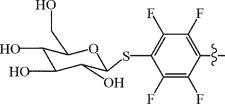

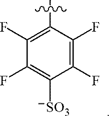

Preferably, R₁, R₂, R₃, R₄, R₅, R₆, R₇, and R₈ are each independently selected from any one of the group consisting of hydrogen, F, Cl, Br, nitro, hydroxyl, sulfonate group, carboxyl, phosphate group, glucosylthio, mannosylthio, fructosylthio, galactosylthio, ribosylthio, xylosylthio, trimethylamino, triethylamino, C₁₋₃ alkyl, C₁₋₃ alkoxyl, halogen-substituted C₁₋₃ alkyl, C₁₋₃ alkyl phosphate group, C₁₋₃ alkyl carboxyl, C₁₋₃ alkyl sulfonate group or their combination.

Further, R₁, R₂, R₃, R₄, R₅, R₆, R₇, and R₈ are each independently selected from any one of the group consisting of hydrogen, F, Cl, Br, nitro, hydroxyl, sulfonate group, carboxyl, glucosylthio, galactosylthio, trimethylamino, triethylamino or their combination.

In some embodimients, R₁, R₂, R₃, R₄, R₅, R₆, R₇, and R₈ are each independently selected from any one of the group consisting of F, sulfonate group, trimethylamine, galactosylthio or their combination.

The porphyrin compounds according to the present invention are one or more selected from the group consisting of the following compounds:

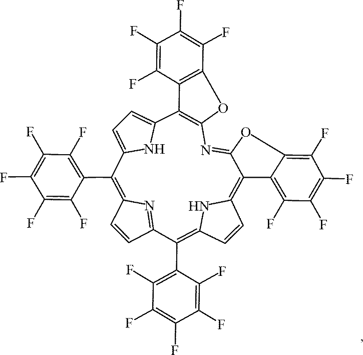

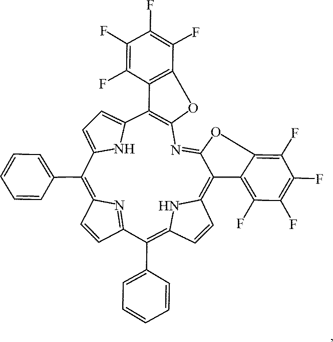

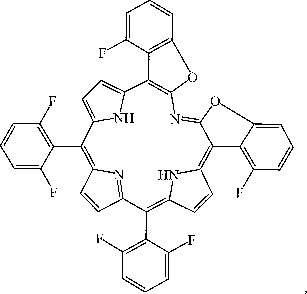

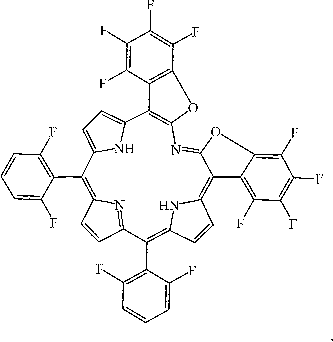

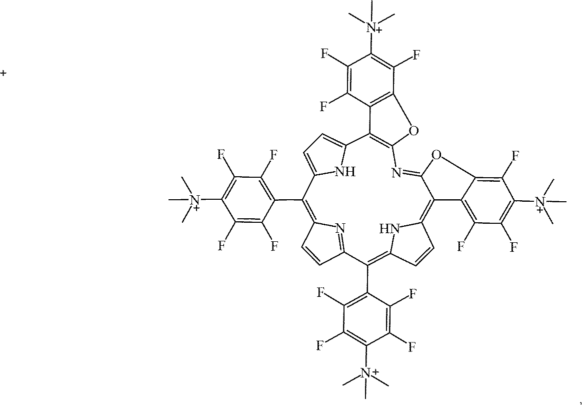

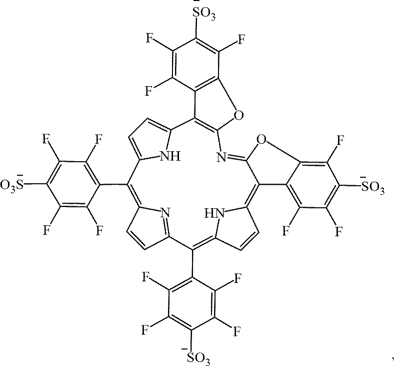

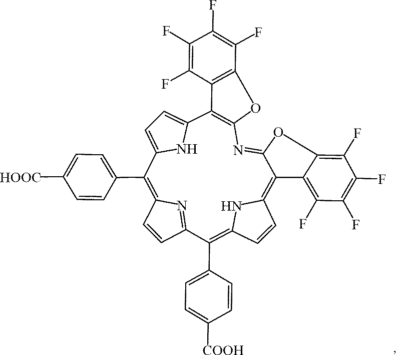

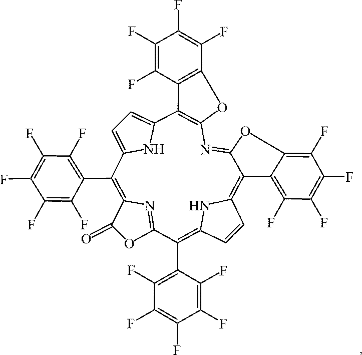

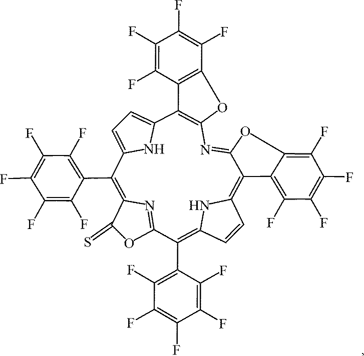

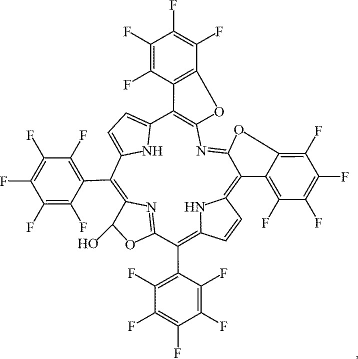

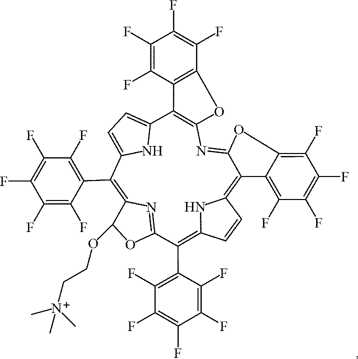

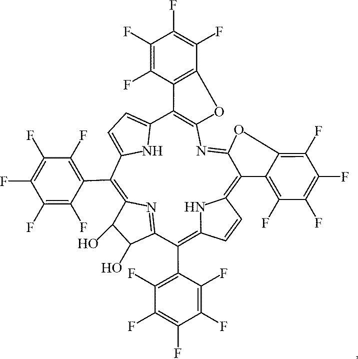

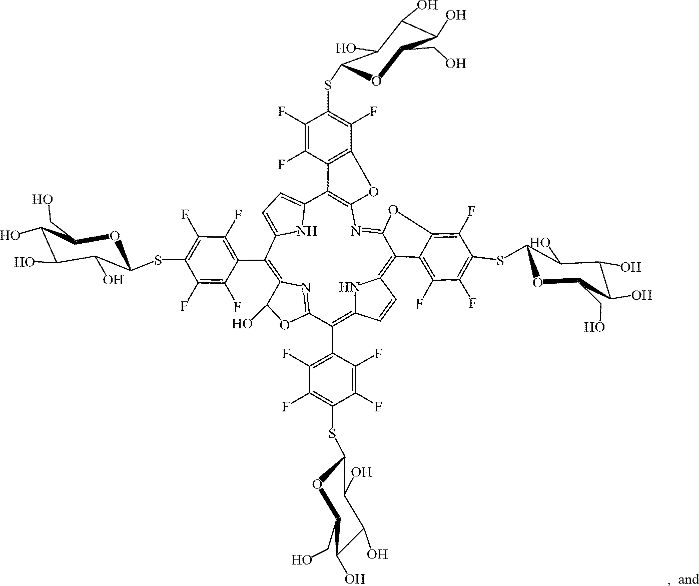

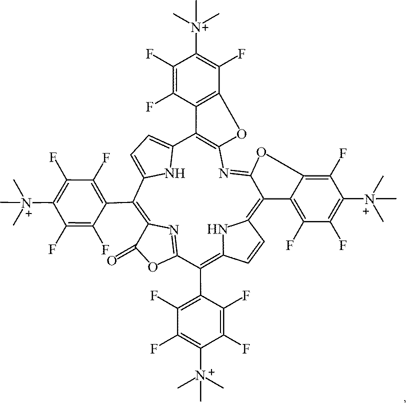

The present invention also provides a method for preparing the porphyrin compound, comprising that: the porphine lactone is reacted with an inorganic base or an organic base in a first organic solvent at 80-200° C. under inert ambient, to produce a first product, which is the porphyrin compound. The reaction equation is as follows.

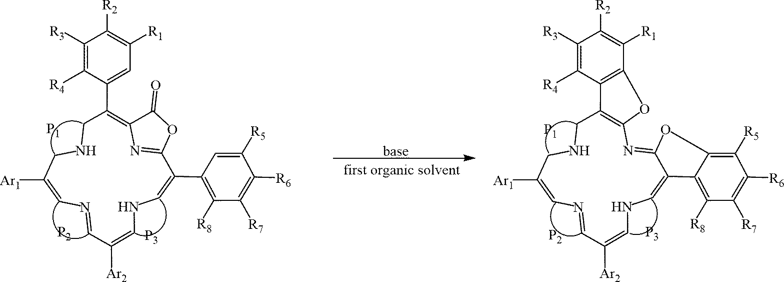

Optionally, the first product is subjected to oxidation, reduction or water-soluble modification reaction in a second organic solvent, to produce a second product, which is also the porphyrin compound.

The oxidation, reduction or water-soluble modification reaction include, for example, lactonization, nucleophilic attack, ionization reaction, or the like.

The first organic solvent is selected from any one or more of the group consisting of decalin, dimethyl sulfoxide, toluene, o-dichlorobenzene, tetrahydrofuran, water, n-hexanol, methanol, acetonitrile, N,N-dimethylformamide, and ethanol, preferably any one or more of the group consisting of tetrahydrofuran, water, acetonitrile, and N,N-dimethylformamide.

The inert ambient refers to a non-oxidizing atmosphere, selected from nitrogen atmosphere, argon atmosphere, or helium atmosphere, preferably, nitrogen atmosphere or argon atmosphere.

The inorganic or organic base includes one or more of potassium carbonate, sodium hydroxide, potassium hydroxide, triethylamine, sodium carbonate, sodium bicarbonate, pyridine, trimethylamine, sodium methoxide, potassium ethoxide, and potassium tert-butoxide.

The second organic solvent is selected from any one or more of the group consisting of water, methanol, chloroform, ethanol, acetonitrile, ethyl acetate, acetone, 1,2-dichloroethane, carbon tetrachloride, tetrahydrofuran, dichloromethane, dimethyl sulfoxide, o-dichlorobenzene, n-hexanol, N,N-dimethylformamide, and toluene, preferably, is any one or more of the group consisting of water, methanol, tetrahydrofuran, 1,2-dichloroethane, carbon tetrachloride, dichloromethane, dimethyl sulfoxide, N,N-dimethylformamide, and chloroform.

The porphyrin compounds according to the present invention have high phototoxicity to a variety of cancer cell lines at both cell and living level. The median lethal concentration of some compounds can be or lower than 1 µM. It shows that the porphyrin compounds are expected to be used as photodynamic therapy drugs in clinical diagnosis and treatment.

The present invention also provides a pharmaceutical composition, comprising the above-mentioned porphyrin compound or the porphyrin compound prepared by the foregoing method as an active ingredient, and pharmaceutically acceptable excipients. The pharmaceutically acceptable salt, solvate, non-covalent bond complex, or prodrug of the porphyrin compound can also be used as the active ingredient of the pharmaceutical composition.

According to the administration routes, the pharmaceutical composition can be formulated into various forms with predetermined dosage of the active ingredient.

When administered through the gastrointestinal tract, the pharmaceutical composition can be used in common dosage forms such as tablets, capsules, oral solutions, oral emulsions and granules.

The pharmaceutical composition according to the present invention can be administered by injection, including intravenous, arterial, intramuscular and spinal cavity injection. The active ingredient can be delivered to the location of lesion by targeted releasing or through a delivery device. The pharmaceutical composition can be used in common dosage forms such as injection solutions, injection emulsions, injection sustained-release solutions, injection suspensions.

The pharmaceutical composition according to the present invention can also be administered externally to the skin by smearing in common dosage forms such as solutions, emulsions, ointments, suspensions, and patches.

In view of the phototoxicity and its characteristics suitable for photodynamic therapy of the porphyrin compound according to the present invention, the pharmaceutical composition is preferably administered by injection or externally use.

According to the dosage form of the pharmaceutical composition, the excipients in the composition should be inactive ingredients that conform to the administration route and have no toxic effects on the human body.

The excipients can be in solid or semi-solid, liquid or gas form. Solid or semi-solid excipients, for example, include sodium chloride, glucose, beeswax, spermaceti, sodium hydroxide, petrolatum, poloxamer, sodium lauryl sulfate, sodium dodecylbenzene sulfonate, cyclodextrin, chitin, lecithin, sodium carboxymethyl cellulose, povidone, starch, magnesium stearate, sodium carboxymethyl starch, talc and methyl paraben. The liquid excipients, for example, include ethylene glycol, water, liquid paraffin, silicone, simethicone, ethanol, peanut oil, phosphoric acid, triethylamine, soybean oil, syrup and glycerin. The gas excipients, for example, include carbon dioxide and nitrogen.

The pharmaceutical composition according to the present invention can be a sterile solution or dispersion system for injection or a sterile powder formulated with sterile water for injection just before use. The composition can be prepared by mixing the active ingredient and excipients such as solvents, isotonicity regulators, surfactants, and antioxidants.

The pharmaceutical composition according to the present invention for example can be a solution, emulsion, ointment or suspension for external use on the skin, and can be prepared by mixing the active ingredient and excipients such as emulsifier, oil-based solvent, water-based solvent.

The pharmaceutical composition should be stable during preparation and storage. Preferably, the amount of the active ingredient in unit dosage is 0.01 mg-20 g.

The porphyrin compound according to the present invention has high phototoxicity, good biocompatibility and high safety, and can be used as a photodynamic therapy drug to treat subcutaneous tumors.

The present invention also provides a use of the porphyrin compound or its pharmaceutically acceptable salt, solvate, non-covalent bond complex or prodrug, and the pharmaceutical composition containing the porphyrin compound as the active ingredient in preparation of drugs for treating subcutaneous tumors.

The subcutaneous tumors include melanoma, sarcoma, fibroma, neurofibroma, lipoma, acne, schwannoma, hemangioma, leiomyoma and lymphangioma.

Depending on the patient’s age, weight, health status, diet, administration route, combination medication, treatment time, etc., the administration dosage may vary individuelly. Typically, in the treatment of the above diseases, the dosage level of the porphyrin compound in the drug applied to each patient is 0.01-500 mg/kg weight/day, or 0.1-20 g/day.

The porphyrin compound has good biocompatibility and high safety, and can effectively inhibit the growth of subcutaneous tumors by injecting or applying to the skin.

The porphyrin compound according to the present invention can significently operate when exposed to deep red to near-infrared irradation, and its absorption and emission wavelengths can cover visible to near-infrared region. Since it can be excited by deep red to near-infrared light (600-1000 nm) having deeper tissue penetration depth, it can be applied in fluorescent labeling, infrared or fluorescent imaging in deep red to near-infrared light region. Preferably, the deep red to near infrared region includes a spectral region in a wavelength range from 650 to 900 nm.

The present invention also provides a use of the porphyrin compound or its pharmaceutically acceptable salt, solvate, non-covalent bond complex or prodrug for fluorescence-labeling and infrared or fluorescence imaging in deep red to near infrared region.

The porphyrin compound according to the present invention has a novel modified structure, and can be derived and modified at multiple sites to achieve biocompatibility modification and functional changes due to different application requirements.

The porphyrin compound has good photodynamic therapy and infrared/fluorescence imaging effects, and is a potential in vivo photodynamic therapy and infrared/fluorescence imaging agent.

### EXAMPLES

### Example 1

Synthesis of molecule 1:

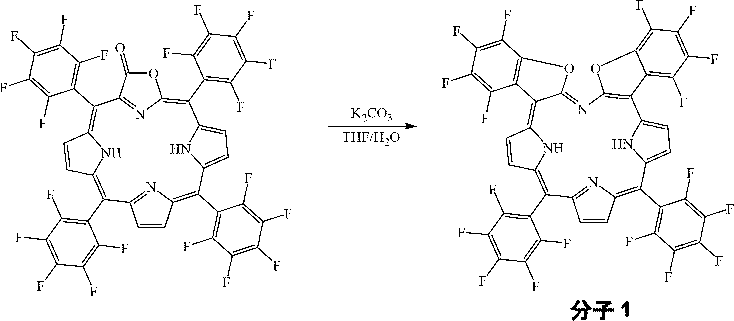

5,10,15,20-tetrapentafluorophenylporphine lactone and potassium carbonate were added in a mixture of tetrahydrofuran and deionized water in a volume ratio of 7:1, and reacted at 200° C. under nitrogen atmosphere to obtain the molecule 1.

The characterization data were shown as follows:

¹H NMR (400 MHz, CDCl₃) δ 9.54 (d, 2 H), 8.67 (d, 2 H), 8.35 (s, 2 H), -0.44 (s, 2 H). ¹⁹F NMR (471 MHz, CDCl₃) δ -137.08 (dd, 4 F), -138.5 (dd, 2 F), -151.21 (t, 2 F), -156.64 (t, 2 F), -160.51 (dd, 2 F), -160.10 (dt, 4 F), -162.13 (t, 2 F). HR-MS (ESI⁺) m/z [M+H]⁺: Calcd for C₄₂H₉F₁₈N₄O₂⁺ 943.0431; found: 943.0446. UV/Vis (CH₂Cl₂, 25° C.): λₘₐₓ(nm) (log ε): 407 (4.69), 440 (4.92), 510 (3.46), 551(3.67), 594 (4.13), 640 (3.86), 696 (4.38).

### Example 2

Synthesis of molecule 2:

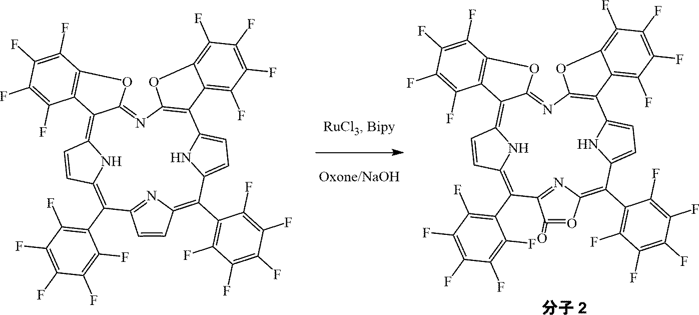

The molecule 1 together with ruthenium trichloride and 2,2-dipyridine were added into 1,2-dichloroethane, and then an aqueous solution of potassium hydrogen persulfate (Oxone) and sodium hydroxide were added dropwise to react at 80° C. under nitrogen atmosphere and obtain the molecule 2.

The characterization data were shown as follows:

¹H NMR (400 MHz, CDCl₃) δ 9.62 (d, 1H), 9.39 (d, 1H), 8.69 (d, 1H), 8.53 (d, 1H), -0.63 (s, 1H), -0.90 (s, 1H). ¹⁹F NMR (471 MHz, CDCl₃) δ -58.48 (dd, 1F), -59.43 (dd, 2F), -59.62 (dd, 1F), -61.24 (dd, 2F), -72.47 (t, 1F), -73.61 (t, 1F), -75.90 (t, 1F), -77.54 (t, 1F), -82.00 (dd, 1F), -82.85 (m, 3F), -83.68 (t, 1F), -83.86 (m, 3F). HR-MS (ESI⁺) m/z [M]: Calcd for C₄₁H₆F₁₈N₄O₄ 960.0102; found: 960.0105. UV/Vis (CH₂Cl₂, 25° C.): λₘₐₓ(nm) (log ε): 410 (4.91), 430 (4.89), 551 (3.82), 594 (4.34), 673 (3.84), 736 (4.56).

### Example 3

Synthesis of molecule 3:

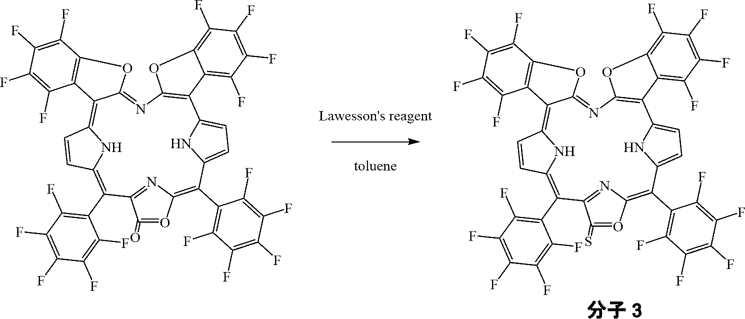

The molecule 2 was mixed with Lawson’s reagent in toluene and reacted at 100° C. under nitrogen atmosphere to obtain molecule 3.

The characterization data were shown as follows:

¹H NMR (400 MHz, CDCl₃) δ 9.56 (d, 2H), 9.33 (d, 2H), 8.65 (d, 1H), 8.53 (d, 1H), -0.11 (s, 1H), -0.28 (s, 1H). ¹⁹F NMR (471 MHz, CDCl₃) δ -136.47 (dd, 1F), -136.99 (dd, 2F), -137.61 (dd, 1F), -139.00 (dd, 2F), -49.97 (t, 1F), -152.13 (t, 1F), -153.57 (t, 1F), -155.14 (t, 1F), -160.31 (m, 3F), -160.89 (t, 1F), -161.24 (t, 1F), -161.40 (m, 2F). HR-MS (ESI⁺) m/z [M+H]⁺: Calcd for C₄₁H₇F₁₈N₄O₃S⁺ 976.9951; found: 976.9950. UV/Vis (CH₂Cl₂, 25° C.): λₘₐₓ(nm) (log ε): 462 (4.65), 491 (5.02), 575 (3.95), 620 (3.65), 698 (3.61), 776 (4.28).

### Example 4

Synthesis of molecule 4:

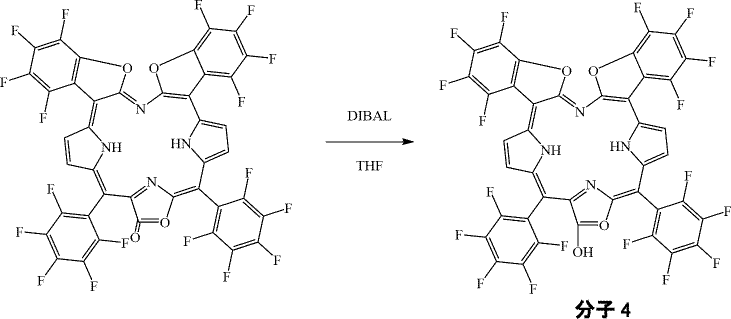

The molecule 2 was mixed with diisobutylaluminum hydride (DIBAL) in tetrahydrofuran and reacted at room temperature (20-40° C.) under nitrogen atmosphere to obtain molecule 4.

The characterization data were shown as follows:

¹H NMR (400 MHz, CDCl₃) δ 9.49 (d, 1H), 9.20 (d, 1H), 8.54 (d, 1H), 8.40 (d, 1H), 8.04 (s, 1H), 7.64 (s, 1H). ¹⁹F NMR (471 MHz, CDCl₃) δ-135.28 (dd, 1F), -136.51 (dd, 1F), -137.34 - -138.15 (m, 3F), -139.73 (dd, 1F), -151.72 (q, 1F), -154.75 (t, 1F), -156.65 (t, 1F), -160.29 (dd, 1F), -160.60 - -161.54 (m, 5F), -162.13 (t, 1F), -162.77 (t, 1F). HR-MS (ESI⁺) m/z [M+H]⁺: Calcd for C₄₁H₉F₁₈N₄O₄⁺ 963.0336; found: 963.0334. UV/Vis (CH₂Cl₂, 25° C.): λₘₐₓ(nm) (log ε): 365 (4.98), 394 (4.91), 541 (4.18), 581 (4.67), 714 (3.89), 790 (4.75).

### Example 5

Synthesis of molecule 5:

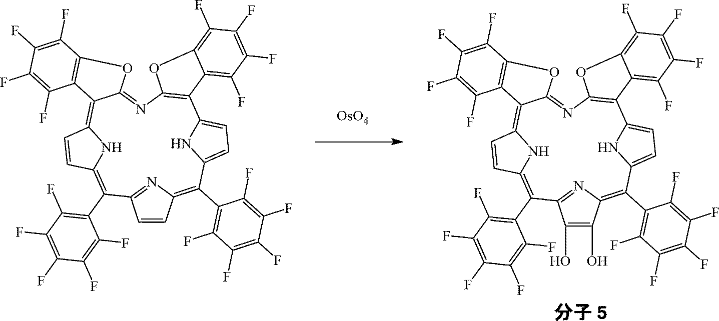 [SMILES: Fc1c(F)c(F)c(-c2c3nc(c(-c4c(F)c(F)c(F)c(F)c4F)c4ccc([nH]4)c4c(nc5oc6c(F)c(F)c(F)c(F)c6c5c5ccc2[nH]5)oc2c(F)c(F)c(F)c(F)c24)C=C3)c(F)c1F ; "OsO4" ; OC1=C(O)c2nc1c(-c1c(F)c(F)c(F)c(F)c1F)c1ccc([nH]1)c1c(nc3oc4c(F)c(F)c(F)c(F)c4c3c3ccc([nH]3)c2-c2c(F)c(F)c(F)c(F)c2F)oc2c(F)c(F)c(F)c(F)c21]

The molecule 1 was mixed with osmium tetroxide in chloroform and reacted at room temperature under nitrogen atmosphere to obtain molecule 5.

The characterization data were shown as follows:

¹H NMR (400 MHz, CDCl₃) δ 9.34 (s, 1H), 8.58 (s, 2H), 8.22 (d, 1H), 7.88 (t, 1H), 7.39 (t, 2H), -1.26 (s, 1H), -0.99 (s, 1H). ¹⁹F NMR (471 MHz, CDCl₃) δ -157.20 (t, 2F), -161.12 (q, 2F), -162.17 (t, 2F), -163.07 (t, 3F), -163.95 (t, 2F). HR-MS (ESI⁺) m/z [M+H]⁺: Calcd for C₄₁H₉F₁₈N₄O₄⁺ 963.0336; found: 963.0334. UV/Vis (CH₂Cl₂, 25° C.): λₘₐₓ(nm) (log ε): 378 (4.95), 390 (4.88), 543 (4.14), 590 (4.62), 710 (3.89), 779 (4.73).

### Example 6

Synthesis of molecule 6:

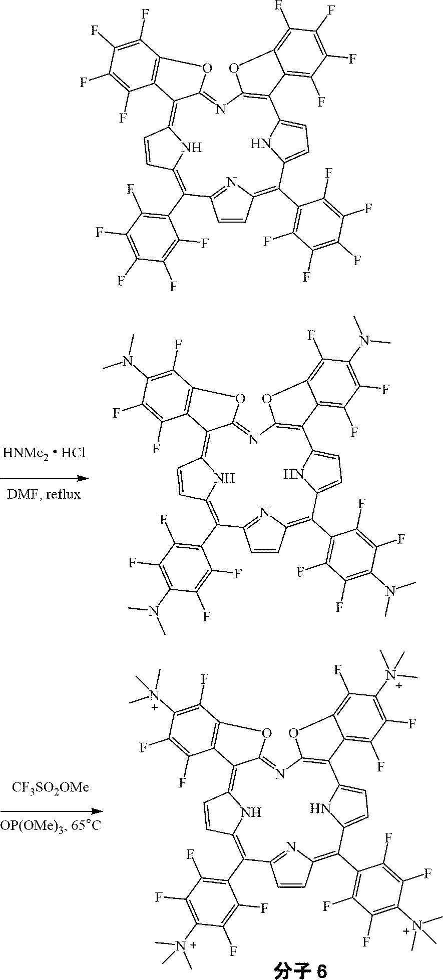

The molecule 1 and dimethylamine hydrochloride were mixed in N,N-dimethylformamide, and reacted at 100° C. under nitrogen atmosphere to obtain an intermediate product. The intermediate product and methyl trifluoromethanesulfonate were mixed in trimethyl phosphate, and reacted at 65° C. under nitrogen atmosphere to obtain molecule 6.

The characterization data were shown as follows:

¹H NMR (400 MHz, D₂O) δ 9.65 (d, 2H), 9.12 (d, 2H), 8.68 (d, 2H), 4.22 (d, 6H). ¹⁹F NMR (471 MHz, D₂O) δ -135.62 (t, 2F), -136.62 (t, 2F), -137.4 (m, 10F), -139.78 (dd, 2F). HR-MS (ESI⁺) m/z [M+H]⁺: Calcd for C₅₄H₄₄F₁₄N₈O₂⁴⁺ 275.5835; found: 275.5835. UV/Vis (H₂O, 25° C.): λₘₐₓ(nm) (log ε): 407 (4.71), 440 (4.92), 510 (3.48), 551 (3.66), 594 (4.15), 640 (3.85), 696 (4.39).

### Example 7

Synthesis of molecule 7:

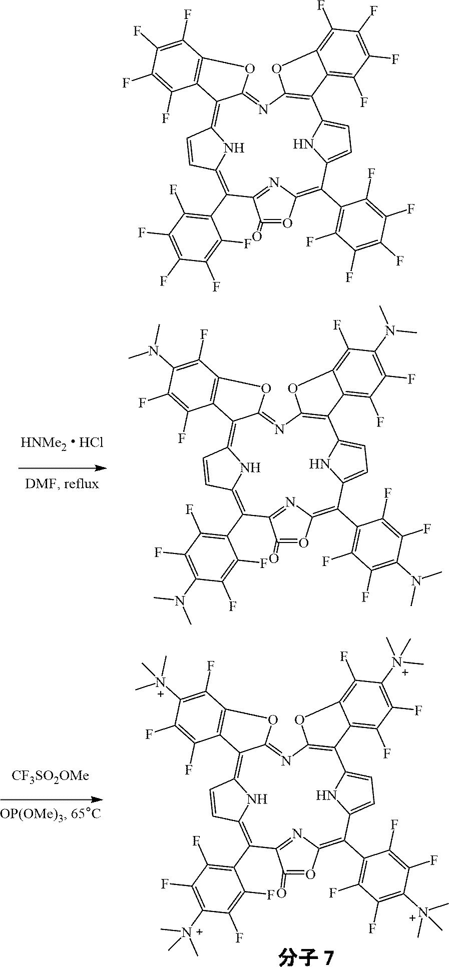

The molecule 2 and dimethylamine hydrochloride were mixed in N,N-dimethylformamide, and reacted at 100° C. under nitrogen atmosphere to obtain an intermediate product. The intermediate product and methyl trifluoromethanesulfonate were mixed in trimethyl phosphate, and reacted at 65° C. under nitrogen atmosphere to obtain molecule 7.

The characterization data were shown as follows:

¹H NMR (400 MHz, CD₃OD) δ 9.76 (d, 1H), 9.46 (d, 1H), 9.15 (d, 1H), 8.85 (d, 1H), 4.21 (s, 36H). ¹⁹F NMR (471 MHz, CD₃OD) δ-135.25 (t, 1F), -136.11 (q, 2F), -136.88 (t, 1F), -137.59 (q, 2F), -138.03 (m, 2F), -138.31 (d, 2F), -139.32 (d, 2F), -140.63 (m, 1F), -141.50 (dd, 1F). HR-MS (ESI⁺) m/z [M+40Tf]²⁺: Calcd for C₅₅H₄₂F₂₀N₈O₁₀S₂²⁺ 709.1073; found: 709.1052. UV/Vis (H₂O, 25° C.): λₘₐₓ(nm) (log ε): 410 (4.90), 430 (4.88), 551 (3.84), 594 (4.32), 673 (3.83), 736 (4.55).

### Example 8

Synthesis of molecule 8:

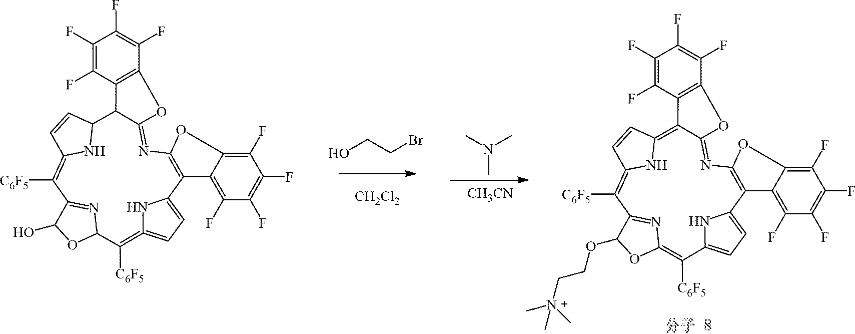

The molecule 4 was reacted with 1-bromoethanol in dichloromethane in the presence of boron trifluoride ether as a catalystat at room temperature, and then subjected to a reflux reaction with trimethylamine in acetonitrile after spin-drying to obtain molecule 8.

The characterization data were shown as follows:

HR-MS (ESI⁺) m/z [M]⁺: Calcd for C₄₆H₂₀F₁₈N₅O₄⁺ 1048.1222; found: 1048.1220. UV/Vis (CH₂Cl₂, 25° C.): λₘₐₓ(nm) (log ε): 365 (4.97), 394 (4.93), 541 (4.20), 581 (4.65), 714 (3.90), 790 (4.75).

### EXPERIMENTAL EXAMPLES

### Experimental Example 1

### Cytophototoxicity Test

The cells used in the experiment included HeLa human cervical cancer cells, HepG2 human liver cancer cells, A375 human malignant melanoma cells, MCF7 human breast cancer cells, and HCT 116 human colon cancer cells. The cells were cultured in DMEM complete medium supplemented with 10% inactivated fetal bovine serum and 1% penicillin-streptomycin at 37° C. under the atmosphere of 5% carbon dioxide.

After trypsinized, the subculture HeLa cells were dispersed in the medium at appropriate concentration. Then, the dispersed HeLa cells were seeded into a poly-D-lysine modified flat-bottom 96-well plate, so that the amount of medium and the number of cells in each well were 200 µL and about 10⁴, respectively, except a group of wells containing medium free of cells for blank control. After culturing the cells in the dark for 24 hours, the medium was removed, and 100 µL of fresh medium and 100 µL of pre-prepared medium solution containing molecule 6 synthesised in Example 6 were added, and then the sample was diluted to a gradient concentration of 0.1-5 µM. After culturing in the dark for another 24 hours, the medium was removed, and each well was rinsed 3 times with PBS (pH=7.4). 100µL of PBS buffer was added to each well, and irradiated

by white light (400-700 nm) with the same light intensity (about 6.5 mW/cm²) under bromo-tungsten lamp for 30 minutes. The PBS in each well was removed and replaced with 200 µL of fresh medium to continue culturing for 24 hours. After that, the medium was removed and the wells were rinsed 3 times with PBS. Then 100 µL of 10% CCK-8 reagent (Cell Counting Kit-8) formulated with medium was added into each well to culture the cells for 2 hours. Meanwhile, 2-(2-methoxy-4-nitrophenyl)-3-(4-nitrophenyl)-5-(2,4-disulfonic acid benzene)-2H-tetrazole monosodium salt (WST-8) can be reduced by living cells together with electronic coupling reagents to yield a yellow product of formazan, accordingly changing the absorbance of the solution at 450 nm, and there was a direct proportional relationship between the change level and the number of living cells. The absorbance change of each well at 450 nm was measued by a microplate reader, and the cell survival rates at various culture concentrations were calculated according to the following equation:

$$\text{CV=} \left({\text{A}}_{\text{s}} - {\text{A}}_{\text{b}}\right) \text{/} \left({\text{A}}_{\text{c}} - {\text{A}}_{\text{b}}\right) \times \text{100\%,}$$

wherein CV refers to the cell survival rate; Aₛ, A_{c} and A_{b} refer to the absorbance of the cells cultured with the compound, the absorbance of the cells cultured without the compound and the absorbance of the blank control, respectively.

The median lethal concentrations IC₅₀ of molecule 6 in different cell lines were calculated according to the cell survival rates at various culture concentrations, and the results were shown as follows:

| cell line | HeLa | HepG2 | A375 | MCF7 | HCT116 |
|---|---|---|---|---|---|
| IC₅₀(µM) | 2.69± 0.1 | 0.80± 0.1 | 4.75± 0.5 | 0.85± 0.1 | 1.52± 0.2 |

It can be seen that, under the condition of light iradiation, the molecule 6 has higher quantum yield of singlet oxygen, and accordingly has higher phototoxicity at the cell level when applied to photodynamic therapy.

### Experimental Example 2

### Animal Test of Photodynamic Therapy

BALB/C nude mice, which were 4-5 weeks old, females, 16-25 grams, were used in the in vivo experiment. Each mouse was inoculated with 100ul (5×10⁶) of A375 human malignant melanoma cells subcutaneously in the right scapula, and the experiment was carried out two weeks later.

The nude mice were randomly individed to control group and skin application group with 6 mice in each group. The control group was only irradiated with light (tungsten lamp, 400-700 nm, 6.5 mW/cm²) without administration, and the 1 mM/20 ul aqueous solution of molecule 6 was administrated to the skin application group in an amount of 10 mg·kg⁻¹ based on the weights of the nude mice. After administrated, the animals were protected from light for 24 hours, and then were each individually irradiated on their tumor sites for 30 minutes a day for consecutive 3 days. The nude mice were not protected from light after the treatment, and were took care in a breeding cage to observe the side effects such as skin phototoxicity appeared on each group of nude mice. After irradiated, the weights of nude mice were measured and their tumor volume were determined with vernier calipers every two days. After 2 weeks of treatment, the lumps were peeled off and weighed.

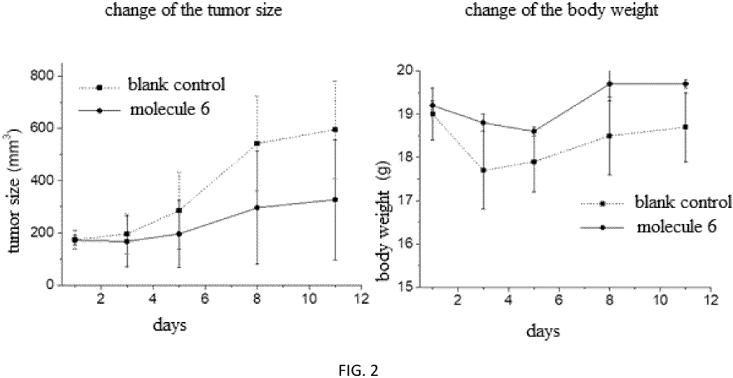

The experiment results of the changes of the tumor size and weight of the control group (blank control) and the skin applicaiton group (molecule 6) were shown in FIG. 2. As can be seen from FIG. 2, the molecule 6 applied on the skin can effectively inhibit the growth of subcutaneous tumors, and the weights of the mice have no significant decrease, while the mice of the control group those were not administered exhibits rapid tumor growth and significant weight loss.

### Experimental Example 3

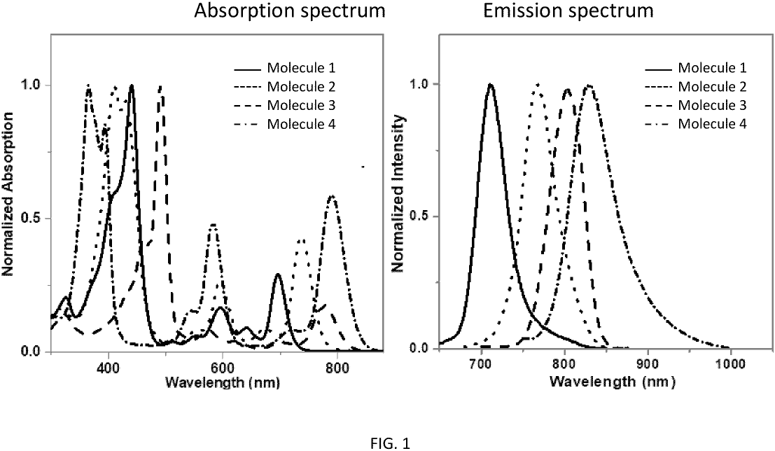

The molecules 1-4 were scanned to ahieve their infrared absorption spectrum and infrared emission spectrum, and the results were shown in FIG. 1. As can be seen from FIG. 1, by modified and derived at multiple sites of the peripheral structures of the porphyrin compounds, the absorption spectrum of the compounds can cover the visible and the near-infrared region. The absorption bands of the compounds in the deep red to near infrared region (650-900 nm) were significantly enhanced. The fluorescence spectra of the compounds excited were also detected in this region, and by varying the modification structure of the compound, its emission wavelength can be red-shifted to 1000 nm, which was contributed to achieve deep tissue penetration depth for infrared imaging or in vivo fluorescence imaging.

### Experimental Example 4

All animal in vivo experiments were carried out by using four-week-old nude mice strictly in compliance with Chinese animal experiment regulations.

IVIS Spectrum fluorescence imaging system was used as the imaging instrument for in vivo fluorescence imaging. The instrument can realize bioluminescence and fluorescence imaging with high sensitivity, and is equipped with 28 high-efficiency filters to cover the full band of 430-850 nm.

During the experiment, 100 uL aqueous solution of 10 uM molecule 8 (containing 1% DMSO) was first injected into the tail vein of the mouse. The mouse was then placed in the imaging instrument under the gas mixture of 2 L/min oxygen and 2% isoflurane to make it anesthetized. The excitation wavelength was 745 nm, the image acquisition wavelength was 840 nm, and the exposure time was set to automatic.

The mouse was euthanized and dissected 4 hours after the compound was injected into the tail vein so as to carry out in vitro organ imaging analysis. The desired organs were took out and imaged in the imaging instrument. Other conditions were same as those of in vivo experiments.

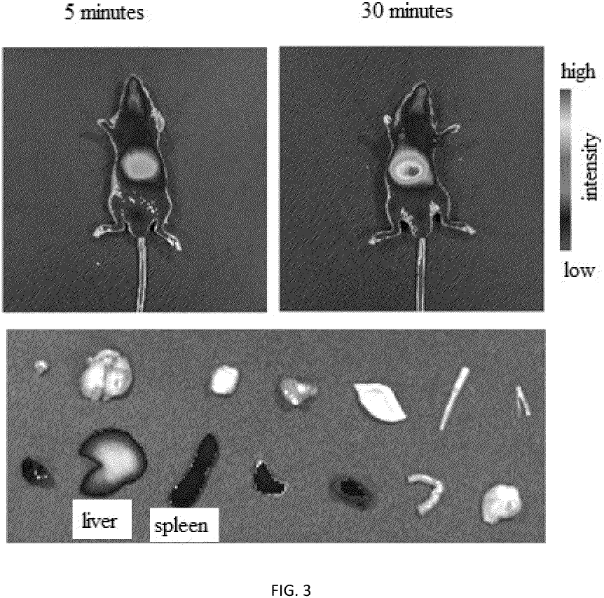

The results were shown in FIG. 3. The compound entered the liver after injected into the mouse. After 30 minutes, the liver can be imaged in a living state with low background interference and weak signals from surrounding tissues. The results of the anatomy experiment and in vivo imaging were consistent, and the compounds were both localized in the liver. It demonstrated that the deep red to near-infrared luminescence property of the compound can effectively reduce the background interference in in vivo fluorescence imaging, and high-resolution fluorescence imaging of specific organs can be achieved even in the non-anatomical state of the living body.

The above describes the present invention in combination with preferred embodiments, but these embodiments are only exemplary and merely serve for illustration. On this basis, various replacements and improvements can be made to the present invention, all of which fall within the protection scope of the present invention.

What is claimed is:

1. A porphyrin compound or its pharmaceutically acceptable salt, solvate, non-covalent complex or prodrug, comprising the following structure: 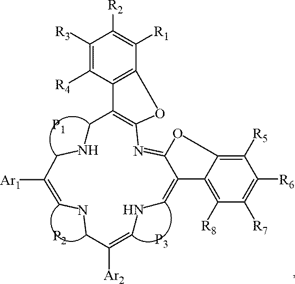
wherein, P₁, P₂ and P₃ are each independently 5-membered ring residues, the terminal carbon atoms of which are connected to N atoms on the 16-membered ring to form a ring; Ar₁ and Ar₂ are substituted or unsubstituted phenyl, aryl or heterocyclic aryl groups; R₁, R₂, R₃, R₄, R₅, R₆, R₇ and R₈ are substituents on benzene rings of the porphyrin compound, each independently selected from any one of the group consisting of hydrogen, halogen, nitro, hydroxyl, amino, mercapto, carboxyl, sulfonate group, phosphate group, cyano group, amide group, C₁-₈ alkyl substituted amino group, substituted or unsubstituted C₁-₁₂ alkyl group, substituted or unsubstituted C₁-₈ alkoxy group, substituted thiol group, C₁-₈ alkyl phosphate group, C₁-₈ alkyl carboxyl group, C₁-₈ alkyl sulfonate group, C₃-₆ alkenyl alkyl group, C₂₋₆ alkenyl group, C₂₋₆ alkynyl alkyl group, C₂₋₆ alkynyl group, C₂₋₆ alkenyloxy group, C₂₋₆ alkynyloxy group, and C₁₋₅ alkanoyl.

2. The porphyrin compound according to claim 1, wherein, 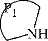 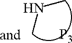
formed by connecting P
₁ and P₃ with N atoms on 16-membered ring of the porphyrin compound are substituted or unsubstituted pyrrole rings; 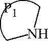 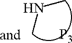
are further each independently selected from one of the group consisting of 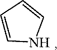 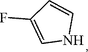 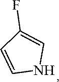 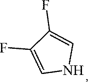 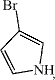 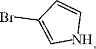 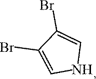 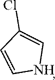 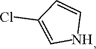 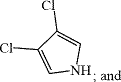 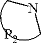
formed by connecting P
₂ with N atom on 16-membered ring of the porphyrin compound is selected from one of the group consisting of 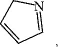 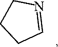 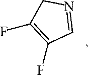 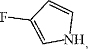 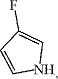 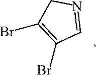 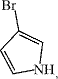 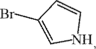 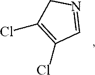 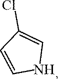 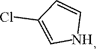 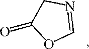 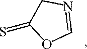 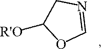 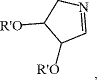 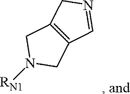 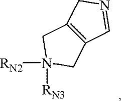
wherein, R′ is selected from hydrogen and trimethylaminoethyl; R_{N1}, R_{N2}, and R_{N3} are each independently selected from the group consisting of hydrogen, C₁₋₄ alkyl, C₁₋₄ alkoxy, halogen-substituted C₁₋₄ alkyl, C₁₋₄ alkyl-substituted amino, or C₁₋₄ alkylthiol.

3. The porphyrin compound according to claim 1, wherein Ar₁ and Ar₂ are substituted phenyl groups containing one or more substituents at sites selected from any one of the group consisting of the following: 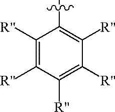 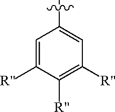 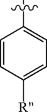 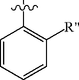 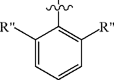 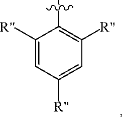
wherein, the substituent groups R″ of Ar₁ and Ar₂ are each independently any one or more selected from the group consisting of hydrogen, halogen, nitro, hydroxyl, mercapto, C₁-₆ alkyl, C₁-₆ alkoxy, halogen substituted C₁-₆ alkyl, C₁-₆ alkyl substituted amino group, substituted thiol group, phosphate group, C₁-₆ alkyl phosphate group, carboxyl, C₁-₆ alkyl carboxyl, sulfonate group, and C₁-₆ alkyl sulfonate group; and R₁, R₂, R₃, R₄, R₅, R₆, R₇, and R₈ are each independently selected from any one of the group consisting of hydrogen, F, Cl, Br, nitro, hydroxyl, sulfonate group, carboxyl, phosphate group, glucosylthio, mannosylthio, fructosylthio, galactosylthio, ribosylthio, xylosylthio, trimethylamino, triethylamino, C₁-₃ alkyl, C₁-₃ alkoxyl, halogen-substituted C₁-₃ alkyl, C₁-₃ alkyl phosphate group, C₁-₃ alkyl carboxyl, C₁-₃ alkyl sulfonate group or their combination.

4. The porphyrin compound according to claim 1, wherein the porphyrin compound is one or more selected from the group consisting of the following compounds: 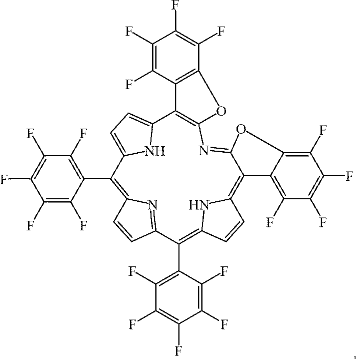 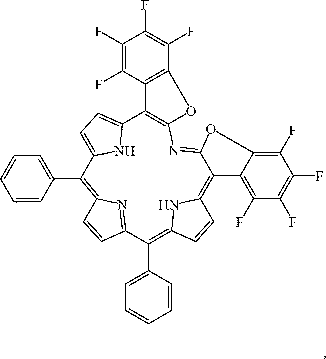 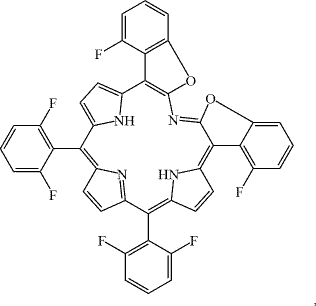 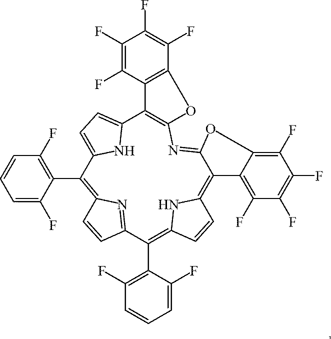 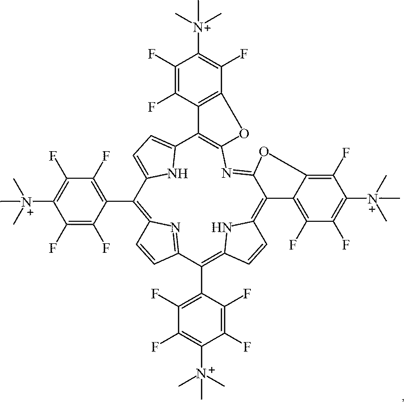 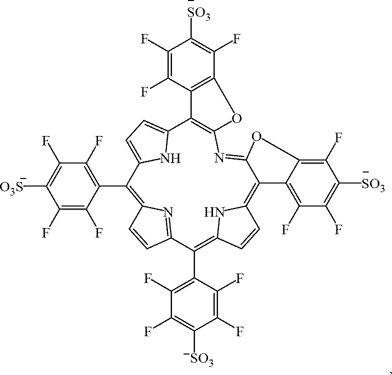 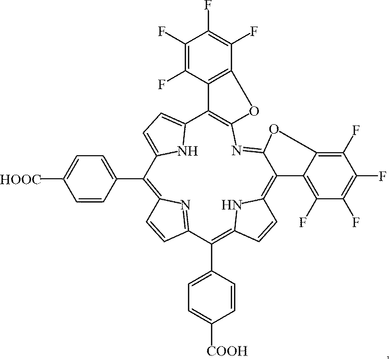 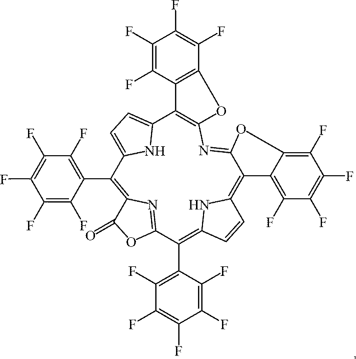 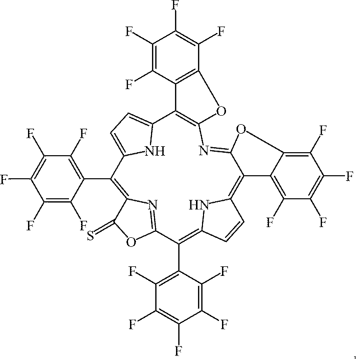 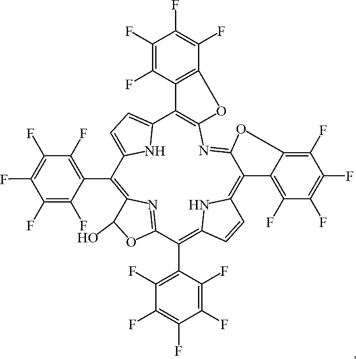 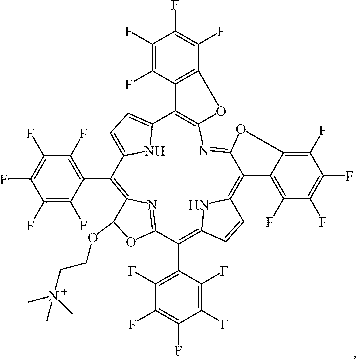 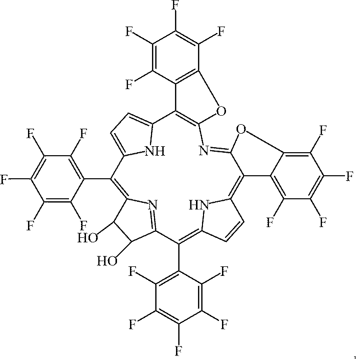 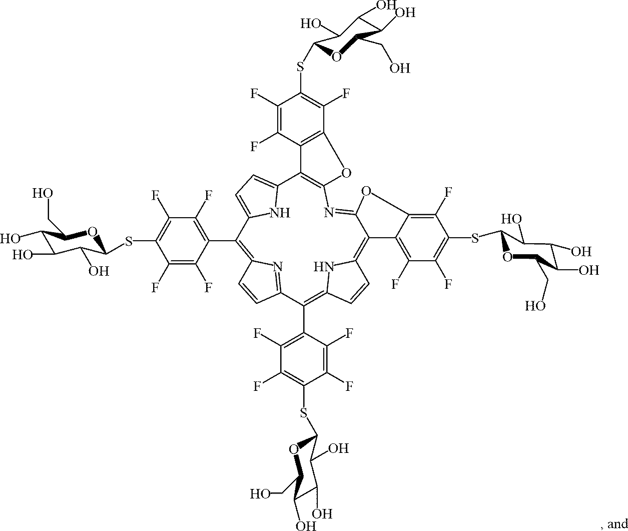 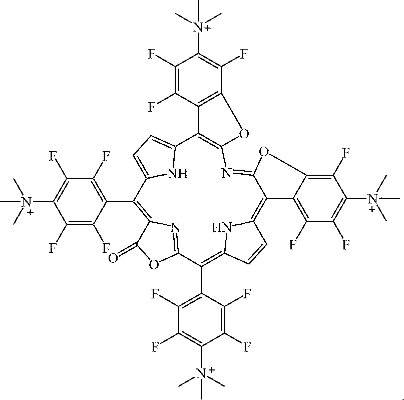
.

5. The porphyrin compound according to claim 1, wherein the porphyrin compound is prepared by the following steps:
the porphine lactone is reacted with an inorganic base or an organic base in a first organic solvent at 80-200° C. under inert ambient, to produce a first product, which is a porphyrin compound;
optionally, the first product is subjected to oxidation, reduction or water-soluble modification reaction in a second organic solvent, to produce a second product, which is also a porphyrin compound.

6. The porphyrin compound according to claim 5, wherein the first organic solvent is selected from any one or more of the group consisting of decalin, dimethyl sulfoxide, toluene, o-dichlorobenzene, tetrahydrofuran, water, n-hexanol, methanol, acetonitrile, N,N-dimethylformamide, and ethanol, preferably any one or more of the group consisting of tetrahydrofuran, water, acetonitrile, and N,N-dimethylformamide; the inert ambient refers to a non-oxidizing atmosphere, selected from nitrogen atmosphere, argon atmosphere, or helium atmosphere.

7. The porphyrin compound according to claim 5, wherein the oxidation, reduction or water-soluble modification reaction include lactonization, nucleophilic attack, and ionization reaction; the second organic solvent is selected from any one or more of the group consisting of water, methanol, chloroform, ethanol, acetonitrile, ethyl acetate, acetone, 1,2-dichloroethane, carbon tetrachloride, tetrahydrofuran, dichloromethane, dimethyl sulfoxide, o-dichlorobenzene, n-hexanol, N,N-dimethylformamide, and toluene.

8. A pharmaceutical composition, comprising the porphyrin compound according to claim 1 as an active ingredient, and pharmaceutically acceptable excipients, wherein the pharmaceutical composition is administrated by injection or externally use; and the amount of the active ingredient in unit dosage of the pharmaceutical composition is 0.01 mg-20 g.

9. A use of the porphyrin compound or its pharmaceutically acceptable salt, solvate, non-covalent bond complex or prodrug according claim 1, and the pharmaceutical composition containing the porphyrin compound as the active ingredient in photodynamic therapy, preferably, in preparation of drugs for treating subcutaneous tumors including melanoma, sarcoma, fibroma, neurofibroma, lipoma, acne, schwannoma, hemangioma, leiomyoma and lymphangioma, wherein the dosage level of the porphyrin compound is 0.01-500 mg/kg weight/day, or 0.1-20 g/day for each patient.

10. The porphyrin compound according to claim 1, wherein the porphyrin compound or its pharmaceutically acceptable salt, solvate, non-covalent bond complex or prodrug is fluorescence-labeling and infrared or fluorescence imaging in deep red to near infrared region.
